(19) United States
(12) Patent Application Publication
(10) Pub. No.: US 2025/0163409 A1
(43) Pub. Date: May 22, 2025

(54) MODIFIED TRANSFER RNA WITH IMPROVED ACTIVITY

(71) Applicant: hC Bioscience, Inc., Boston, MA (US)

(72) Inventors: David H. Altreuter, Wayland, MA (US); Yosef Landesman, Brookline, MA (US)

(21) Appl. No.: 18/777,627

(22) Filed: Jul. 19, 2024

Related U.S. Application Data
(63) Continuation of application No. PCT/US2023/011223, filed on Jan. 20, 2023.
(60) Provisional application No. 63/301,930, filed on Jan. 21, 2022.

(51) Int. Cl. C12N 15/11

(52) U.S. Cl. CPC C12N 15/11; C12N 2310/315; C12N 2310/321; C12N 2310/344; C12N 2320/51

(57) ABSTRACT

The present invention is related at least in part to modified transfer RNAs (tRNAs) and related methods and compositions.

### RELATED APPLICATIONS

This application claims the benefit of priority under 35 U.S.C. § 119(e) of U.S. Provisional Application Ser. No. 63/301,930, filed on Jan. 21, 2022, the entire contents of which are incorporated herein by reference.

### BACKGROUND

DNA molecules carry genetic information in the form of the sequence of the nucleotide bases that make up the DNA polymer. Only four nucleotide bases are utilized in DNA: adenine, guanine, cytosine, and thymine. This information, in the form of codons of three contiguous bases is transcribed into messenger RNA (mRNA), and then translated by transfer RNA (tRNA) and ribosomes to form proteins. Four nucleotide bases are utilized in RNA: adenine, guanine, cytosine, and uracil. The genetic code is the relation between a triplet codon and a particular amino acid. Sixty-four possible codon triplets form the genetic code, where three stop (also called “terminating” or “nonsense”) codons provide a signal to the translation machinery (cellular ribosomes) to stop protein production at the particular codon. The other sixty-one codon triplets (also called “sense codons”) correspond to one of the 20 standard amino acids.

### SUMMARY OF THE INVENTION

tRNA delivered to the body in unmodified form can be more susceptible to degradation in vivo, and therefore, may have reduced efficiency and/or function for efficacious therapeutic benefit. Provided herein are approaches to provide modified tRNA that can have enhanced activity (e.g., efficacy).

In one aspect, compositions comprising tRNAs with such modifications are provided herein as are related compositions and methods of their use. In some embodiments of any one of the compositions or methods provided herein, the tRNAs have been modified at at least one of any one of the specific locations provided herein and/or as provided in any one of the examples provided herein. Such tRNAs can have prolonged circulation half-life and/or different affinity to other molecules and/or increased presence or accumulation at one or more target tissue or cell types and/or increased uptake at one or more target tissues or cell types and/or increased functional readout at one or more target tissues or cell types.

In some embodiments of any one of the compositions or methods provided herein, the tRNAs have been modified such that at least one of any one of the specific locations provided herein can have or has a chemical modification. In some embodiments of any one of the compositions or methods provided herein, the modified tRNAs have 2′ sugar modifications (e.g., 2′-O-methyl (2′-OMe) modifications). In some embodiments of any one of the compositions or methods provided herein, the tRNAs have been modified such that at least one of any one of the specific locations provided herein can have or has a backbone modification. In some embodiments of any one of the compositions or methods provided herein, the modified tRNAs have phosphorothioate modifications. In some embodiments of any one of the compositions or methods provided herein, the modified tRNAs have 2′ sugar modifications (e.g., 2′-O-methyl (2′-OMe) modifications) and phosphorothioate modifications.

In some embodiments of any one of the compositions or methods provided herein, the tRNA is any one of the modified tRNAs provided herein, in labeled or unlabeled form. In some embodiments of any one of the compositions or methods provided herein, the tRNA is any one of the modified tRNAs provided herein, with or with a label and/or quencher.

In some embodiments, compositions comprising the modified tRNA(s) are provided. In some embodiments, oligonucleotides encoding the modified tRNA(s) are provided. In some embodiments, compositions comprising the oligonucleotide(s) provided herein are provided. The oligonucleotide may be an expression cassette or any vector from which the modified tRNA(s) may be produced.

In some embodiments, the modified tRNAs or compositions provided herein can be used for administration to a subject, such as for treatment of the subject. In some embodiments, the modified tRNAs or compositions provided herein can be used for restoring protein translation in a subject, for altering a resulting protein, etc. The modified tRNAs or compositions provided herein can be used to correct for a nonsense or missense mutation. The modified tRNAs or compositions provided herein can be used to replace a stop codon or other codon with an alternate amino acid in a protein or portion thereof.

Methods of producing the modifications are also provided herein. Such methods can comprise any one or more of the specific steps provided herein.

Active tRNA molecules can have a conserved secondary and tertiary structure that is a component of its activity, including, for example, specific recognition by aminoacyl tRNA synthetase, specific recognition by elongation factors, fit with ribosomal a, p and e sites (aminoacyl, peptidyl, and exit sites), and/or anticodon interaction with mRNA codons, etc.

In some embodiments, the modification is at one or more positions in the tRNA sequence. In some embodiments, the one or more positions is in the acceptor stem of the tRNA.

Aspects of the present disclosure relate to a composition comprising any one of the tRNA(s) described herein.

In some embodiments, any one of the improved effects of the tRNA, such as the targeting of the tRNA, is improved compared with a control tRNA without the modification(s).

Aspects of the present disclosure relate to a method comprising administering to a subject in need thereof any one of the tRNAs described herein or any one of the compositions described herein, and, optionally, in an amount effective to achieve any one or more of the improved effects provided herein, such as in the subject. In some embodiments, the administration is systemic or local administration. In some embodiments, the subject is a human.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 provides a table of the genetic code.

FIG. 2 shows the general four-arm structure of tRNAs comprising a T-arm, a D-arm, an anticodon arm, and an acceptor stem (or arm). These regions may also be referred to as ‘loops’ throughout.

FIG. 3 shows the activity of example modified tRNAs.

### DETAILED DESCRIPTION

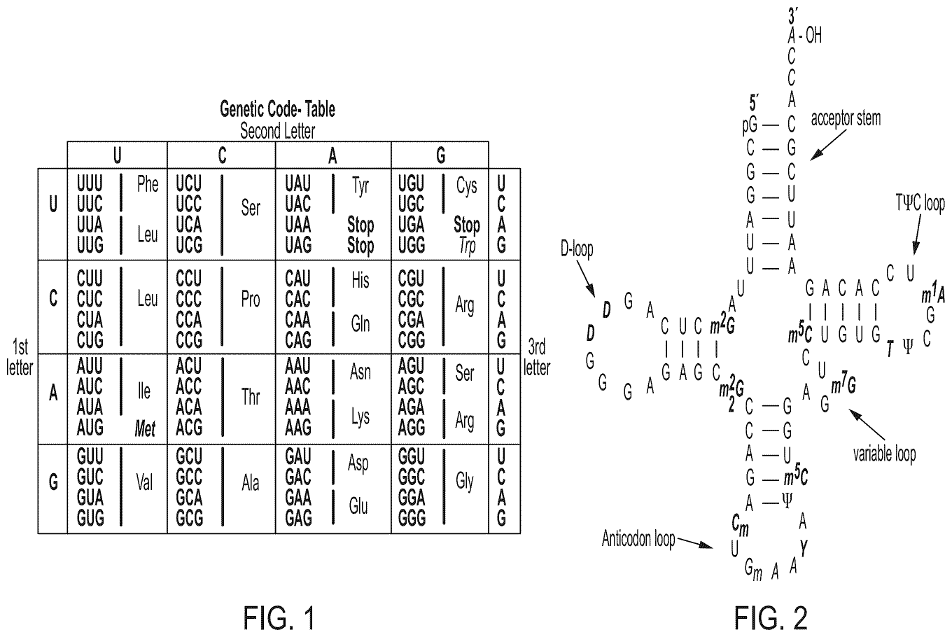

Transfer RNAs (tRNAs) are decoders of DNA and RNA “blueprints.” DNA transcription results in messenger RNAs (mRNAs) that encode primary amino acid structures that may be modified post-transcriptionally and that, upon interaction with ribosomes and tRNAs, eventually become folded or unfolded proteins. Once an mRNA engages with a ribosome, tRNAs deliver amino acids to the ribosome and form a chain of amino acids based on the code of the mRNA (FIG. 1).

tRNAs have a general four-arm structure comprising a T-arm, a D-arm, an anticodon arm, and an acceptor stem or arm (FIG. 2). The T-arm is made up of a “T-stem” and a “TΨC loop.” Any one of the tRNAs provided herein can comprise this four-arm structure. The tRNAs are approximately 100 nucleotides in length, in some embodiments, and can be readily introduced into cells. Engineered tRNAs have the potential to correct defects in damaged or mutated mRNA molecules by delivering a correct amino acid where an incorrect amino acid may normally be delivered. Engineered tRNAs may also have the potential to reduce the amount of protein in a cell by delivering an incorrect amino acid wherein a correct amino acid may normally be delivered, thereby rendering the consequent amino acid defective and unable to fold and/or susceptible to protein degradation.

Engineered or therapeutic tRNAs delivered to the body in unmodified form without benefit of a delivery agent, vector, electro-mechanical facilitation, permeation-enhancing agent or the like is expected to quickly become substantially degraded and lack meaningful capacity for cell uptake, thereby presenting very limited potential for therapeutic benefit. An immune response directed at the tRNA may also occur. Accordingly, modified, engineered tRNAs have the potential to be delivered to a cell effectively, which may increase therapeutic efficacy.

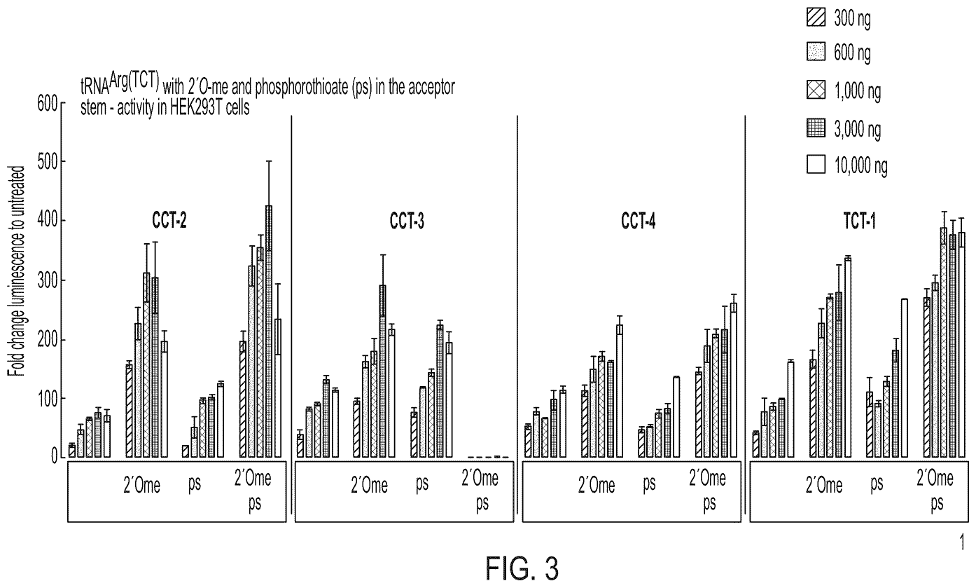

Provided herein are compositions and methods related to a technology based on modifications of tRNAs. In some embodiments, a tRNA can be engineered to comprise one or more backbone modifications. In some embodiments, one or more modified nucleotides are located at one or more positions of the tRNA. In some embodiments, a tRNA can be engineered to comprise one or more nucleoside modifications. In some embodiments, a tRNA can be engineered to comprise one or more nucleoside modification and one or more backbone modifications. It has been found that such tRNAs (such as those of the Examples and FIG. 3) have increased activity as compared to unmodified tRNA.

In some embodiments, backbone modifications include, but are not limited to, phosphorothioates, phosphotriesters, methyl phosphonates, short chain alkyl or cycloalkyl intersugar linkages or short chain heteroatomic and heterocyclic intersugar linkages. In some embodiments, the modified tRNA can comprise phosphorothioate backbones; heteroatom backbones, such as methylene(methylimino) or MMI backbones; amide backbones; morpholino backbones; or peptide nucleic acid (PNA) backbones. In some embodiments, a modified backbone may include any modification(s) that is/are suitable for the present disclosure.

In some embodiments, modified nucleosides can comprise 2′ O-methyl, 2′-fluoro, 2′-amino and/or 2′-hydroxyl. In some embodiments, modified nucleosides can comprise any one or more of 2′ O-methyl, a 2′-fluoro, 2′-amino and 2′-hydroxyl. For example, a tRNA may be modified to comprise a 2′ O-methyl and/or a 2′-fluoro modification. In some embodiments, a 2′ O-methyl, a 2′-fluoro, and/or a 2′-amino can be substituted in the 2′-hydroxyl position of the ribose sugar. In some embodiments, a modified nucleoside can include any modification(s) that is/are suitable for the present disclosure.

As used herein, “phosphorothioate substitutions” refer to chemical modifications of the tRNA backbone where a single nonbridging oxygen of the phosphate is replaced with a sulfur atom. Phosphorothioate substitutions can stabilize a tRNA molecule by protecting it from hydrolysis.

As used herein, “2′-fluoro or 2′-O-methyl substitutions at the 2′-hydroxyl position of the ribose sugar” refers to 2′-sugar modifications such as 2′-fluoro or 2′-O-methyl substitutions at the 2′-hydroxyl position of the ribose sugar in RNA that can, in some embodiments, increase the stability of the RNA in solution. Without wishing to be bound by any theory, a 2′-fluoro substitution at the 2′-hydroxyl position of a ribose sugar in a RNA backbone can be generated when the 2′-hydroxyl position (2′-OH) of the ribose sugar is substituted by a fluorine atom. A 2′-O-methyl substitution at the 2′-hydroxyl position of a ribose sugar in a RNA backbone can be generated when the 2′-hydroxyl position (2′-OH) of the ribose sugar is substituted by an O-methyl (0-Me, or O—CH3) group.

In some embodiments, the tRNAs described herein may further comprise one or more covalent conjugations and the one or more modifications can include those made covalently.

In some embodiments, the present disclosure provides a composition that comprises one or more tRNAs as described herein.

The present disclosure provides methods of increasing the half-life of a tRNA, such as in vivo. The present disclosure provides methods of increasing stability of a tRNA, such as in vivo. In some embodiments, the methods described herein prolong circulation of the tRNA, such as in vivo, by reducing susceptibility to nucleases. In some embodiments, prolonged circulation of the tRNA can impact association affinity of the tRNA with other molecules, so that the tRNA results in improved efficacy.

In some embodiments, increasing the half-life or the stability of a tRNA in vivo comprises modifying a nucleoside and/or backbone of the tRNA. In some embodiments, the half-life or the stability of the tRNA is increased compared with a control. As used herein, a “control” refers to a tRNA that is not so modified. A control may be the same tRNA but without the modifications as provided herein. Comparisons of half-life, stability, or any other features can be assessed in vitro as known by the ordinarily skilled artisan or in vivo with the use of a test subject.

In some embodiments, the half-life or stability of the modified tRNA is increased by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control tRNA.

In some embodiments, compositions comprising the modified tRNA(s) are provided. In some embodiments, oligonucleotides encoding a modified tRNA is provided. In some embodiments, compositions comprising the oligonucleotide(s) provided herein are provided. The oligonucleotide may be an expression cassette or any vector from which the tRNA may be produced.

The present disclosure provides methods of treating a subject in need thereof, such as one with a disease or disorder. In some embodiments, the methods comprise administering to a subject a tRNA or a composition as disclosed herein. In some embodiments, the disease or disorder may include a genetic disease or disorder or a hyperproliferative disease or disorder. In some embodiments, the disease or disorder may be cancer. The tRNAs can also be used to disrupt protein expression and/or function, preferably the protein expression and/or function of a protein encoded by an oncogene or tumor suppressor gene. The tRNAs can also be used where cell survival or mobility would be a benefit. The tRNAs can also be used to restore protein expression and/or function. In some embodiments, the disease or disorder may be any disease or disorder that can benefit from a tRNA treatment.

In some embodiments, the disease or disorder is improved (e.g., less severe) in the subject administered the tRNA by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control tRNA.

As used herein, a “genetic disease or disorder” is a disease or disorder that is caused by mutations that are inherited or that arise within a subject's genetic code or that predisposes a subject to a disease or disorder. Any one of the compositions or methods provided herein can be for treating or preventing a genetic disease or disorder.

As used herein, “hyperproliferative disease or disorder” refers to any disease or disorder where there is an abnormally high rate of proliferation of cells by rapid division, substantial overproliferation, etc. Certain embodiments of the present disclosure provide a method of treating a disease or disorder, such as a hyperproliferative disease or disorder in a subject, such as a mammal. In certain embodiments, the mammal is human. Certain embodiments of the present disclosure provide a use of a tRNA or composition as described herein to prepare a medicament useful for treating a disease or disorder, such as a hyperproliferative disease or disorder, in a subject, such as a mammal, such as a human. In certain embodiments, the therapy has potential use for the treatment/management of a disease or disorder, such as a hyperproliferative disease or disorder, including tumors, cancers, and neoplastic tissue, along with non-neoplastic or non-malignant hyperproliferative disorders. In certain embodiments, the hyperproliferative disease or disorder is cancer.

The present disclosure provides methods for improving cellular uptake of the tRNA by administering the tRNA to a subject. In some embodiments, the cellular uptake is improved by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control tRNA.

The present disclosure provides methods for increasing on-target activity by administering the tRNA to a subject. In some embodiments, the on-target activity is increased by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control tRNA.

The present disclosure provides methods for reducing immunogenicity that is caused by the tRNA by administering the tRNA to a subject. In some embodiments, the immunogenicity is reduced by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control tRNA.

The tRNAs and nucleotide sequences encoding the tRNAs can be generated synthetically.

Any one of the tRNAs provided herein can have the sequence of any tRNA, such as those provided herein or otherwise known in the art, but with a modification as provided herein. Nucleotide sequences encoding human tRNAs are known and generally available to those of skill in the art through sources such as GenBank. The structure of tRNAs is highly conserved, and tRNAs can be functional across species. Thus, bacterial or other eukaryotic tRNA sequences are also potential sources for tRNAs of the invention, but with an anticodon as provided herein. The determination of whether a particular tRNA is functional as desired, such as in a desired mammalian cell, can be ascertained as described herein or through other experimentation that will be apparent to one of ordinary skill in the art with the benefit of the teachings provided herein. In an embodiment of any one of the compositions or methods provided herein, the tRNA sequences may be any of the sequences provided in PCT/US2018/059065, WO2019/090154, WO2019/090169, and Lueck et al., Nature Communications 10, 822, 2019, the sequences of which are incorporated herein by reference, for example, but with an anticodon as provided herein.

The tRNA(s) may be in any form suitable (e.g., any suitable recombinant plasmid that comprises a heterologous nucleic acid sequence) for delivery to a target cell, either in vitro or in vivo. The heterologous nucleic acid sequence encodes a gene product (e.g., a tRNA) of interest for the purposes of, for example, any one of the uses provided herein including disease treatment, and may, optionally, be in the form of an expression cassette. The term “recombinant” refers to a polynucleotide which either does not occur in nature or is linked to another polynucleotide in an arrangement not found in nature. The term “heterologous,” as used herein refers to a nucleic acid sequence obtained or derived from a genetically distinct entity from the rest of the entity to which it is being compared.

The tRNA(s) or composition as described herein can be delivered to a cell in vivo or in vitro. Administration to the cell can be accomplished by any means, including simply contacting the cell. The contact with the cells can be for any desired length of time. The cells can include any desired cell in humans as well as other large (non-rodent) mammals, such as primates, horse, sheep, goat, pig, and dog. Any one of the subjects provided herein can be a human or other mammal. The term “mammal” includes, but is not limited to, humans, mice, rats, guinea pigs, monkeys, dogs, cats, horses, cows, pigs, and sheep.

Methods for determining the amount of any of the modified tRNA molecules described herein that has been effectively delivered to a cell in vitro, as compared to the amount of control tRNA molecules delivered to a cell in vitro are also provided or otherwise understood in the art. In some embodiments, the amount of modified and control tRNA molecules that have been effectively delivered to a cell in vitro can be determined by sequencing, measurement of protein production rates, protein analysis, or any other technique otherwise understood in the art. In some embodiments, verification of increased levels of modified tRNA molecules, as compared to control tRNA molecules, is indicative of such activity in vivo.

In some embodiments, administration can be systemic. In some embodiments, administration can be local. In some embodiments, administration can be direct delivery to the selected organ, oral, inhalation, intraocular, intravenous including facial vein injection and retroorbital injection, intracerebroventricular (ICV), intracisterna magna (ICM) injection, intramuscular, intrathecal, intracranial, subcutaneous, intradermal, intratumoral, and other parental routes of administration. Routes of administration may be combined, if desired. In some embodiments, the tRNA as disclosed herein can be administered via any route that is appropriate for the present disclosure.

Suitable methods for the delivery and introduction into a subject are also provided or otherwise understood in the art. In one embodiment, pharmaceutical compositions will comprise sufficient genetic material to produce a therapeutically effective amount of the tRNA of interest. The tRNAs or composition can be delivered in an effective amount and into a cell, such as with endogenous tRNA synthetase. A tRNA synthetase is considered to be “endogenous” to a cell if it is present in the cell into which a tRNA is introduced. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

In some embodiments, the tRNA or composition can be formulated in a pharmaceutical composition. The pharmaceutical compositions may also contain a pharmaceutically acceptable excipient. Such excipients include any pharmaceutical agent that may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

As is apparent to those skilled in the art in view of the teachings of this specification, an effective amount of the tRNAs or compositions provided may be empirically determined. Administration can be effected in one dose, continuously or intermittently, throughout the course of treatment. Methods of determining the most effective means and dosages of administration may vary with the composition of the therapy, target cells, and the subject being treated, etc. Single and multiple administrations can be carried out with the dose level and pattern being selected by the treating physician.

Vehicles including water, aqueous saline, artificial CSF, or other known substances can be employed. To prepare a formulation, the purified composition can be isolated. The composition may then be adjusted to an appropriate concentration and packaged for use.

As used herein, the terms “treat” and “treatment” refer to both therapeutic treatment and measures that can alleviate symptoms or provide some benefit to a subject, wherein the object is to prevent or slow down (lessen) an undesired physiological change or disorder, such as the growth, development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition, disease or disorder.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, malignancies, etc.

The tRNA or composition can be administered so as to result in a reduction in at least one symptom associated with a disease or disorder, such as a genetic disease or disorder or a hyperproliferative disease or disorder (e.g., cancer). The amount administered will vary depending on various factors including, but not limited to, the composition chosen, the particular disease, the weight, the physical condition, and the age of the mammal. Such factors can be readily determined by the clinician employing animal models or other test systems that are known to the art.

Administration of the tRNA or composition may be continuous or intermittent, depending, for example, upon the recipient's physiological condition, whether the purpose of the administration is therapeutic, and other factors known to skilled practitioners. The administration of the tRNA or composition may be essentially continuous over a preselected period of time or may be in a series of spaced doses.

One or more suitable unit dosage forms having the tRNA or composition of the invention may be formulated and can be administered by a variety of routes. When the agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. Pharmaceutical formulations containing the tRNA or composition can be prepared by procedures known in the art using well-known and readily available ingredients. The tRNA or composition can also be formulated as solutions appropriate for administration. The pharmaceutical formulations of the tRNA or composition can also take the form of an aqueous or anhydrous solution or dispersion, or alternatively the form of an emulsion or suspension.

Thus, the tRNA or composition may be formulated for administration and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in a suitable vehicle, e.g., sterile, pyrogen-free water, before use. It will be appreciated that the unit content of active ingredient or ingredients contained in an individual dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

Any of the compositions provided herein can be placed in contact with, administered to or introduced into a cell with genetic transfer methods, such as transfection. Thus, any of the compositions provided herein can be included with or in a gene delivery vehicle. The gene delivery vehicle can be any delivery vehicle known in the art and can include naked nucleic acid that is facilitated by a receptor and/or lipid mediated transfection.

The tRNAs or compositions provided herein can be contacted with cells or delivered or administered to a subject within a particle, such as a nanoparticle. A particle, such as a nanoparticle, can be, but is not limited to, lipid-based nanoparticles (also referred to herein as lipid nanoparticles, i.e., nanoparticles where the majority of the material that makes up their structure are lipids) and/or particles with a combination of nanomaterials. The particles may be a variety of different shapes, including but not limited to spheroidal, cuboidal, pyramidal, oblong, cylindrical, toroidal, and the like. In some embodiments, particles, such as nanoparticles, may comprise one or more lipids. In some embodiments, particles, such as nanoparticles, may comprise liposomes. In some embodiments, particles, such as nanoparticles, may comprise a lipid bilayer. In some embodiments, particles, such as nanoparticles, may comprise a lipid monolayer. In some embodiments, particles, such as nanoparticles, may comprise a micelle.

The tRNAs or compositions provided herein can be contacted with cells or delivered or administered to a subject with an extracellular vesicle or exosome. Generally, exosomes are nano-sized extracellular vesicles (EVs) (30-150 nm in diameter) which can be formed and released by many mammalian cells. The EVs or exosomes can be loaded with an agent of interest, for example by pre-treatment of cells with the agent and then isolation of loaded EVs or exosomes. EVs or exosomes can be derived from human embryonic kidney cells, bone marrow stem cells, immature dendritic cells, red blood cells as well as from milk. EVs or exosomes can be isolated and purified with a number of different techniques. Such methods include, but are not limited to ultracentrifugation, ultrafiltration, size exclusion chromatography (SEC), precipitation with polymers, and separation by affinity-based methods, such as immunomagnetic-based isolation.

In an embodiment of any one of the compositions or methods provided herein, a nucleic acid sequence encoding a tRNA is in a closed-end form, such as in a plasmid, nanoplasmid or minicircle. In an embodiment of any one of the compositions or methods provided herein, the nucleic acid sequence encoding a tRNA is in the form of a minicircle or microthread.

The term “minicircle”, as used herein, refers to small circular DNA fragments that are largely or completely free of non-essential prokaryotic elements. Minicircles include circular forms of DNA without prokaryotic elements and/or in which prokaryotic elements have been removed. Minicircles can be from a parental plasmid where bacterial DNA sequences have been excised. The minicircle may be in the form of any suitable recombinant plasmid that comprises a heterologous nucleic acid sequence to be delivered to a target cell, either in vitro or in vivo. The preparation of minicircles have been described in the art (e.g., in Nehlsen et al., Gene Ther. Mol. Biol. 10: 233-244, 2006; and Kay et al., Nature Biotechnology. 28: 1287-1289, 2010). The preparation can, for example, follow a two-step procedure: (i) production of a ‘parental plasmid’ (bacterial plasmid with eukaryotic inserts); and (ii) induction of a site-specific recombinase at the end of this process. These steps can be followed by the excision of prokaryotic vector parts via recombinase-target sequences and recovery by capillary gel electrophoresis.

As a nonlimiting example, a minicircle may be produced as follows. An expression cassette, which comprises the polynucleotide coding sequence along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the polynucleotide; this minicircle DNA vector is devoid of any bacterial DNA sequences. The second circular DNA sequence contains the remaining vector sequence, including the bacterial sequences and the sequence encoding the recombinase. The minicircle DNA containing the polynucleotide sequence can then be separately isolated and purified. In some embodiments, a minicircle DNA vector may be produced using plasmids similar to pBAD. ϕ.C31.hFIX and pBAD. ϕ.C31.RHB. See, e.g., Chen et al. (2003) Mol. Ther. 8:495-500, or as otherwise provided herein.

Examples of recombinases that may be used for creating a minicircle include, but are not limited to, Streptomyces bacteriophage #31 integrase, Cre recombinase, and the X integrase/DNA topoisomerase IV complex. Each of these recombinases catalyzes recombination between distinct sites. For example, #31 integrase catalyzes recombination between corresponding attP and attB sites, Cre recombinase catalyzes recombination between loxP sites, and the λ integrase/DNA topoisomerase IV complex catalyzes recombination between bacteriophage λ attP and attB sites.

Published US Application 20170342424 also describes a system making use of a parent plasmid which is exposed to an enzyme which causes recombination at recombination sites, thereby forming a (i) minicircle including the polynucleotide sequence and (ii) miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination.

Removal of prokaryotic sequences ideally should be efficient, using the smallest possible excision site, while creating supercoiled DNA minicircles which consist solely of gene expression elements under appropriate—preferably mammalian—control regions. Some techniques for minicircle production use bacterial phage lambda (λ) integrase mediated recombination to produce minicircle DNA. See, for example, Darquet, et al. 1997 Gene Ther 4(12): 1341-9; Darquet et al. 1999 Gene Ther 6(2): 209-18; and Kreiss, et al. 1998 Appl Micbiol Biotechnol 49(5):560-7).

Kits for producing minicircle DNA are known in the art and are commercially available (System Biosciences, Inc., Palo Alto, Calif.). For example, a MC-EasyTM (Cat #MN920A-1, SBI System Biosciences) Minicircle DNA production kit can be used to obtain high-quality minicircle DNA. Information on minicircle DNA is provided in Dietz et al., Vector Engineering and Delivery Molecular Therapy (2013); 21 8, 1526-1535 and Hou et al., Molecular Therapy-Methods & Clinical Development, Article number: 14062 (2015) doi:10.1038/mtm.2014.62. More information on Minicircles is provided in Chen Z Y, He C Y, Ehrhardt A, Kay M A. Mol Ther. 2003 September; 8(3):495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21(1):131.

In some embodiments, the closed-end form is a supercoiled helix. DNA supercoiling refers to the amount of twist in a particular DNA strand. Supercoiled DNA can be positively supercoiled DNA or negatively supercoiled DNA. As used herein, “supercoiled DNA” refers to a DNA molecule, or fragment of a DNA molecule, wherein one or both DNA strands comprise increased twisting compared to the amount of twisting in a reference state known as “relaxed B-form” DNA. In a “relaxed” double-helical segment of DNA, the two strands twist around the helical axis once every 10.4-10.5 base pairs of sequence. A given DNA strand may be “positively supercoiled” or “negatively supercoiled” (i.e., more or less tightly wound). Supercoiling creates twist strain in the DNA strand. The amount of a strand's supercoiling affects a number of biological processes, such as compacting DNA and regulating access to the genetic code (which strongly affects DNA metabolism and possibly gene expression). Certain enzymes (e.g., topoisomerases) are capable of increasing or decreasing the amount of twisting (e.g., supercoiling) in a DNA strand in order to facilitate functions such as DNA replication and transcription. If a DNA segment under twist strain is closed into a circle by joining its two ends, and then allowed to move freely, it may form a supercoiled structure. Examples of supercoiled structures of circular DNA molecules include, but are not limited to a figure-eight structure, a plectonemic structure, or a toroidal structure.

Electroporation may also be used.

In an embodiment of any one of the compositions or methods provided herein, an oligonucleotide encoding any one of the modified tRNAs is provided. In an embodiment of any one of the compositions or methods provided herein, an oligonucleotide described herein further comprises a promoter. Such an oligonucleotide may be comprise in a vector.

The promoter characteristically has a specific nucleotide sequence necessary to initiate transcription. In some embodiments, an oligonucleotide as provided herein comprises a nucleic acid sequence capable of directing expression of a particular nucleotide sequence in an appropriate cell, which may include a promoter operably linked to the nucleotide sequence of interest that may also be operably linked to termination signals (or terminator). An oligonucleotide as provided herein may be in a recombinant form useful for heterologous expression. In certain embodiments, the promoter is a regulatable promoter. In certain embodiments, the promoter is a constitutive promoter. The promoter to drive expression of the sequence encoding a tRNA to be delivered can be any desired promoter, selected by known considerations, such as the level of expression of a nucleic acid functionally linked to the promoter and the cell type in which the vector is to be used. Promoters can be an exogenous or an endogenous promoter.

In an embodiment of any one of the compositions or methods provided herein, promoters may be between (35-105 bp in size). The promoters may be any known promoters, including native tRNA leader sequences, which sequences may be ˜50-60 bp in size. In an embodiment of any one of the compositions or methods provided herein, the promoter may be reduced-sequence or re-configured promoters. Example promoter sequences that may be comprise in any one of the oligonucleotides or vectors provided herein include, but are not limited to, any one of the sequences provided herein or otherwise known in the art.

Optionally, the oligonucleotides or vectors provided herein may further include additional sequences (e.g., 3′ tRNA tail or trailer sequences). Such sequences may be between 2-20 bp in length. They may include natural sequences or engineered variants with 3-10 consecutive “T” residues.

The present disclosure also provides a cell containing a tRNA, oligonucleotide, or vector described herein. The cell may be mammalian, such as human. According to one aspect, a cell expression system is provided. The expression system comprises a cell and an oligonucleotide or vector as provided herein. Expression cassettes include, but are not limited to, plasmids, viral vectors, and other vehicles for delivering heterologous genetic material to cells. The cell expression system can be formed in vivo.

In some embodiments of any one of the tRNAs provided herein, the tRNA is one not found in nature. In some embodiments of the foregoing, such a tRNA is engineered or modified from that found in nature. In some embodiments of the foregoing, such a tRNA is a recombinant tRNA. In other embodiments of any one of the tRNAs provided herein, the tRNA is selected for improved activity as provided herein and can be used in any one of the methods provided herein.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Evaluation of Modified tRNA Activity

### Materials and Methods

### Design and Generation of Modified ACE-tRNAs

Human ACE-tRNA sequences were used as the backbone for generation of modified tRNAs, tRNAArg(CCT)-2, tRNAArg(CCT)-3, tRNAArg(CCT)-4, tRNAArg(TCT)-1. Specifically, the anticodon sequence of each of the tRNAs was altered to be complementary to the UGA stop codon. The ribose of nucleotides at the beginning of the acceptor stem (N1, N2, N3 and/or N4) of the sequences was modified with a 2′-O-methyl group. The phosphate backbone between such nucleotides was changed to a phosphorothioate backbone. Some sequences contained both 2′-O-ME modifications and phosphorothioate. All sequences were synthesized by Trilink BioTechnologies and delivered as lyophilized powder. The tRNAs were resuspended in water to concentration of 10 mg/ml.

### Cell Culture

HEK293T cells stably expressing pNluc-UGA (nanoluciferase with a UGA stop codon at position 160) (from Dr. Chris Ahern, University of Iowa) were cultured in Dulbecco's Modified Eagle Medium (Gibco cat #10566-016) that contains Glutamax and 4.5 g/L Glucose and was supplemented with 10% FBS and 1× pencilin/streptomycin. The cells were cultured in T75 tissue culture treated flasks in a 5% CO2 incubator at 37° C. For cell passaging, cells were first washed with PBS, then 3 ml of 0.25% Trypsin-EDTA were added to the flasks, which were then incubated at 37° C. for˜5 minutes. The flasks were then returned form the incubator and 7 ml of DMEM media was added. The cells were counted and split for different purposes.

Transient tRNA Transfection

24 hours before transfection HEK293T cells were seeded in white, clear bottom tissue culture treated 96—well plates at 10,000 cells/well in 100 μl/well of DMEM media containing Glutamax and 4.5 g/L Glucose that was supplemented with 10% FBS with no antibiotics. The cells were transfected with the tRNA variants at 300 ng/ml, 600 ng/ml, 1,000 ng/ml, 3,000 ng/ml, and 10,000 ng/ml in triplicate but avoiding the wells on the perimeter of the plate, using Lipofectamine 3000 (Invitrogen Cat. #L3000001). 24 hours after transfection the media was changed to DMEM media containing Glutamax and 4.5 g/L Glucose and supplemented with 10% FBS and 1× Penicilin/Streptomycin.

### Nanoluciferase Detection Assay

48 hours post transfection, the 96-well plates were removed from the incubator and the activity of the tRNA was measured in the following way. 100 μl of reconstituted nano-glo dual-luciferase reporter assay system (Promega Cat. #N1130) (200 μl substrate added to 10 ml buffer) was added to each well and the plates were left on the bench for 10 minutes to allow full lysis. The luminescence was then measured using the Varioskan LUX plate reader (Thermo Fisher cat #VL0000D0). The fold change luminescence to untreated signal was obtained by averaging the values from each triplicate treatment and dividing that value by averaged background measurement from the untreated wells.

### Results

It was surprising found that the addition of 2′-O-methyl group to first nucleobases of the ACE-tRNA significantly improved its activity. In addition, it was found that incorporating a phosphorothioate backbone into the chain of such nucleobases also improved its activity. Finally, combining both 2′-O-methyl groups and incorporating phosphorothioate can improve the activity even further.

### EQUIVALENTS

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A transfer RNA (tRNA), wherein the tRNA comprises one or more nucleoside modifications and/or one or more backbone modifications.

2. The tRNA of claim 1, wherein the one or more nucleoside modifications comprise a 2′ sugar modification (e.g., 2′ O-methyl).

3. The tRNA of claim 1, wherein the one or more backbone modifications comprise a phosphorothioate internucleotide substitution.

4. The tRNA of claim 1, wherein the modifications are in the acceptor stem of the tRNA, optionally, in one or more of the first four nucleotides/nucleobases (e.g., in all four).

5. An oligonucleotide comprising a sequence that encodes the modified tRNA of claim 1.

6. The oligonucleotide of claim 5, wherein the oligonucleotide is DNA or RNA.

7. The oligonucleotide of claim 5, further comprising a promoter and/or a terminator.

8. The oligonucleotide of claim 5, wherein the oligonucleotide is in a plasmid or closed-end form.

9. A vector, such as a viral vector, comprising the oligonucleotide of claim 5.

10. A composition comprising the tRNA of claim 1, further comprising a pharmaceutically acceptable carrier.

11. A particle, such as a nanoparticle, that comprises the tRNA of any one of claim 1.

12. The particle of claim 11, wherein the particle is a liposome or lipid nanoparticle.

13. A composition comprising the particle of claim 11, or a population of such particles, optionally, further comprising a pharmaceutically acceptable carrier.

14. A cell comprising the tRNA of claim 1.

15. A method of increasing the activity of a tRNA (in vitro or in vivo), wherein the method comprises modifying the tRNA to comprise one or more nucleoside modifications and/or one or more backbone modifications.

16. A method of increasing the half-life or stability of a tRNA (in vitro or in vivo), wherein the method comprises modifying the tRNA to comprise one or more nucleoside modifications and/or one or more backbone modifications.

17. The method of claim 15, wherein the one or more nucleoside modifications comprise a 2′ O-methyl.

18. The method of claim 15, wherein the one or more backbone modifications comprise a phosphorothioate internucleotide substitution.

19. The method of claim 15, wherein the modifications are in the acceptor stem of the tRNA.

20-22. (canceled)

23. A method comprising administering to a subject in need thereof, the tRNA of claim 1.

24-25. (canceled)
